(19) United States
(12) Patent Application Publication
(10) Pub. No.: US 2002/0187998 A1
(43) Pub. Date: Dec. 12, 2002

(54) Local ophthalmic agent for treatment of ocular inflammation

(75) Inventor: Ryuji Ueno, Potomac, MD (US)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: Sucampo AG, Zug (CH)

(21) Appl. No.: 10/120,515

(22) Filed: Apr. 12, 2002

Related U.S. Application Data
(60) Provisional application No. 60/283,169, filed on Apr. 12, 2001.

(51) Int. Cl. A61K 31/4745; A61K 31/407

(52) U.S. Cl. 514/291; 514/411

(57) ABSTRACT

An agent for topical ophthalmic treatment of ocular inflammatory disease of a human contains a tricyclo compound or a pharmaceutically acceptable salt thereof as an active ingredient in a concentration of 0.01%-0.1%. The agent shows superior ocular anti-inflammatory effects by topical administration in a low dose to the eye of a human having an ocular inflammatory disease. The agent is effective for symptoms caused by ocular inflammatory diseases. The agent is also effective for subjects in whom conventional anti-inflammatory agents show no improving effect and is effective for subjects for whom other anti-inflammatory agents cannot be used. The agent is effective by topical administration to the eye one to four times daily.

### BACKGROUND OF THE INVENTION

1. Field of the Invention

The present invention relates to an agent for topical ophthalmic treatment of ocular inflammatory diseases containing a tricyclo compound as its active ingredient.

2. Discussion of the Background

Ocular inflammatory diseases include many forms of ocular disorders with the accompanying pain depending on the position of inflammation. Ocular inflammatory diseases include uveitis, conjunctivitis, cyclitis, scleritis, episcleritis, optic neuritis, retrobulbar optic neuritis, keratitis, blepharitis, corneal ulcer, conjunctival ulcer, etc. Further, ocular inflammatory diseases may be caused by various ocular disorders, an ophthalmic operation or a physical injury to the eye.

Symptoms of ocular inflammatory diseases include itching, flare, edema, ulcer, etc.

Patients with ocular inflammatory diseases account for more than half of all the patients with ocular diseases. Accordingly, agents having ocular anti-inflammatory effects play an important role in the medical scene. Today, steroid drugs and nonsteroidal drugs are mainly used for ocular inflammatory diseases.

Steroid drugs, which have excellent effects on ocular inflammatory diseases, are clinically indispensable drugs. However, whether they are administered systemically or topically, they have the risk of serious side effects. Such side effects include, for example, steroid glaucoma, infectious eye diseases, steroidal cataract, etc. Especially, patients with chronic ocular inflammatory diseases have a high risk of such side effects. For the specific patients having an already increased intraocular pressure (e.g., glaucoma patients), such side effects can never be acceptable. Under these circumstances, it has been strongly desired to develop a nonsteroidal ocular anti-inflammatory agent.

Presently, several nonsteroidal anti-inflammatory agents for internal use have been launched. However, in topically administered agents for treating ocular inflammatory diseases, especially eye drops, the agent needs to have characteristics that satisfy requirements unique and necessary to eye drops, such as improvement of water solubility, release of topical irritations on the eye, good transition to the eye tissues, etc., in addition to anti-inflammatory effects. Therefore, it has not been easy to develop a nonsteroidal agent that satisfies these requirements and is effective for ocular inflammatory diseases.

Besides, in the case of eye drops, the amount administrable at one time is small compared to that for agents for internal use. Thus, in many cases, even when the agent is effective as an internal agent it does not show sufficient effect in ocular instillation or it is necessary to administer the agent frequently (at least four times a day). Therefore, it is desired to develop non-steroidal anti-inflammatory eye drops having greater effects in a small amount.

### SUMMARY OF THE INVENTION

Accordingly, an object of the present invention is to provide a non-steroidal ocular anti-inflammatory agent having superior ocular anti-inflammatory effects in a small amount with high safety.

It is known that FK506 and cyclosporins are effective for the treatment of allergic diseases such as allergic conjunctivitis, vernal conjunctivitis, atopic dermatitis, etc. (e.g., WO92/19278).

However, it has not yet been reported that a tricyclo compound such as FK506 can show superior ocular anti-inflammatory effects when topically administered in low doses to the eye of a human suffering from an ocular inflammatory disease. This effect is shown even in subjects in whom conventional anti-inflammatory agents show no improving effect and for subjects for whom other anti-inflammatory agents cannot be used (e.g., steroid contraindication).

The present inventor has conducted intensive studies and has found that a tricyclo compound shows superior ocular anti-inflammatory effects by topical administration in low doses to the eye of a human suffering from ocular inflammatory disease. Further, the present inventor has found that this type of tricyclo compound for topical ophthalmic treatment is effective for symptoms caused by ocular inflammatory diseases such as itching, flare, edema, ulcer, etc. Furthermore, the present inventor has found that the tricyclo compound for topical ophthalmic treatment is effective even for subjects for whom conventional anti-inflammatory agents (e.g., steroid and cyclosporins) show no improving effect and is effective even for subjects for whom other anti-inflammatory agents cannot be used (e.g., steroid contraindication). In this way, the present invention has been completed.

Accordingly the present invention provides the following.

(1) A method for treating ocular inflammatory diseases, comprising topical administration of an agent for topical ophthalmic treatment comprising a tricyclo compound as shown by the following general formula (I), or its pharmaceutically acceptable salt, to the eye of a human in need of treatment of ocular inflammatory diseases in a concentration of 0.01%-0.1%:

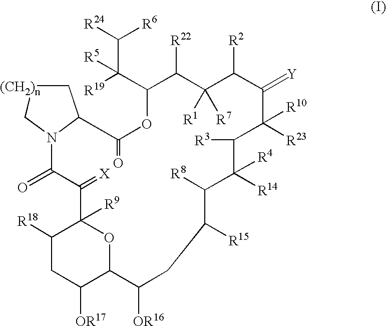

wherein adjacent pairs of R¹ and R², R³ and R⁴, and R⁵ and R⁶ each independently

a) consist of two adjacent hydrogen atoms, wherein R² is optionally alkyl, or

b) form another bond optionally between carbon atoms binding with the members of said pairs;

R⁷ is hydrogen atom, hydroxy, protected hydroxy or alkyloxy, or may form oxo with R¹;

R⁸ and R⁹ each independently show hydrogen atom or hydroxy;

R¹⁰ is hydrogen atom, alkyl, alkyl substituted by one or more hydroxy, alkenyl, alkenyl substituted by one or more hydroxy or alkyl substituted by oxo;

X is oxo, (hydrogen atom, hydroxy), (hydrogen atom, hydrogen atom), or a group of the formula —CH₂O—;

Y is oxo, (hydrogen atom, hydroxy), (hydrogen atom, hydrogen atom), or a group of the formula N—NR¹¹R¹² or N—OR¹³;

R¹¹ and R¹² each independently show hydrogen atom, alkyl, aryl or tosyl;

R¹³, R¹⁴, R¹⁵, R¹⁶, R¹⁷, R¹⁸, R¹⁹, R²² and R²³ each independently show hydrogen atoms or alkyl;

R²⁴ is an optionally substituted ring that may contain one or more hetero atom(s); and n is 1 or 2,

in addition to the meaning noted above, Y, R¹⁰ and R²³ may have, together with the carbon atom they bind with, a saturated or unsaturated 5 or 6-membered heterocyclic group containing nitrogen atom, sulfur atom and/or oxygen atom, the heterocyclic group being optionally substituted by one or more group(s) selected from alkyl, hydroxy, alkyloxy, benzyl, a group of the formula —CH₂Se(C₆H₅), and alkyl substituted by one or more hydroxy, or its pharmaceutically acceptable salt.

(2) The method as described in (1) wherein the tricyclo compound is FK506.

(3) The method as described in (1) wherein the topical administration to the eye is one to four times a day.

(4) The method as described in (1) wherein the agent for topical ophthalmic treatment is an eye drop or eye ointment.

(5) The method as described in (1) wherein the ocular inflammatory diseases are selected from a group consisting of uveitis, conjunctivitis, cyclitis, scleritis, episcleritis, optic neuritis, retrobulbar optic neuritis, keratitis, blepharitis, corneal ulcer, conjunctival ulcer and symptoms caused by them; the ocular inflammatory disease caused by the ocular disorders; the ocular inflammatory diseases after an ophthalmic operation; and the ocular inflammatory diseases caused by a physical injury.

(6) The method as described in (1) wherein the treatment of the ocular inflammatory diseases is targeted for treating itching on the eye.

(7) The method as described in (1) wherein the treatment of the ocular inflammatory diseases is targeted for treating flare on the eye.

(8) The method as described in (1) wherein the treatment of the ocular inflammatory diseases is targeted for treating edema on the eye.

(9) The method as described in (1) wherein the treatment of the ocular inflammatory diseases is targeted for treating ulcer on the eye.

(10) The method as described in (1) comprising administration to a human in whom other ocular anti-inflammatory agents show no improving effect.

(11) The method as described in (10) wherein the other ocular anti-inflammatory agents are cyclosporins and/or steroid drugs.

(12) The method as described in (1) comprising administration to a human for whom other ocular anti-inflammatory agents cannot be used.

(13) The method as described in (12) wherein the other ocular anti-inflammatory agents are steroid drugs.

(14) An agent for topical ophthalmic treatment of a human for ocular inflammatory diseases, comprising a tricyclo compound as shown by the following general formula (I) or its pharmaceutically acceptable salt as the active ingredient in the concentration of 0.01%-0.1%.

(15) The agent as described in (14) wherein the tricyclo compound is FK506.

(16) The agent as described in (14) wherein the topical ophthalmic treatment by administration to the eye is performed one to four times a day.

(17) The agent as described in (14), which is an eye drop or eye ointment.

(18) The agent as described in (14) wherein the ocular inflammatory diseases are selected from a group consisting of uveitis, conjunctivitis, cyclitis, scleritis, episcleritis, optic neuritis, retrobulbar optic neuritis, keratitis, blepharitis, corneal ulcer, conjunctival ulcer and symptoms caused by them; the ocular inflammatory disease caused by the ocular disorders; the ocular inflammatory diseases after an ophthalmic operation; and the ocular inflammatory diseases caused by a physical injury.

(19) The agent as described in (14) wherein the topical ophthalmic treatment is targeted for treating itching on the eye.

(20) The agent as described in (14) wherein the topical ophthalmic treatment is targeted for treating flare on the eye.

(21) The agent as described in (14) wherein the topical ophthalmic treatment is targeted for treating edema on the eye.

(22) The agent as described in (14) wherein the topical ophthalmic treatment is targeted for treating ulcer on the eye.

(23) The agent as described in (14), which is used for administration to a human in whom other ocular anti-inflammatory agents show no improving effect.

(24) The agent as described in (23) wherein the ocular anti-inflammatory agents are cyclosporins and/or steroid drugs.

(25) The agent as described in (14), which is used for administration to a human for whom other ocular anti-inflammatory agents cannot be used.

(26) The agent as described in (25) wherein the ocular anti-inflammatory agents are cyclosporins and/or steroid drugs.

(27) A use of a tricyclo compound as shown by the following general formula (I) or its pharmaceutically acceptable salt for manufacturing an agent for topical ophthalmic treatment of a human for treating ocular inflammatory diseases characterized in that said agent for treatment comprises said tricyclo compound in the concentration of 0.01%-0.1%.

### BRIEF DESCRIPTION OF THE DRAWINGS

FIG. 1 is a graph showing that itching decreases by instillation of FK506 eye drop.

### DESCRIPTION OF THE PREFERRED EMBODIMENTS

The present invention provides an agent for topical ophthalmic treatment of a human for ocular inflammatory diseases, comprising a tricyclo compound as shown by the following general formula (I) or its pharmaceutically acceptable salts as the active ingredient in the concentration of 0.01%-0.1%:

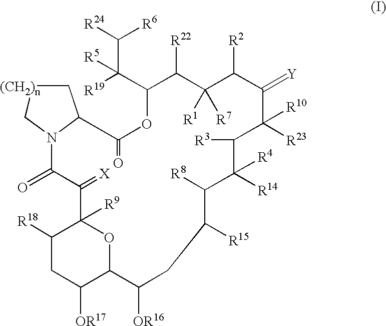

wherein adjacent pairs of R¹ and R², R³ and R⁴, and R⁵ and R⁶ each independently

a) consist of two adjacent hydrogen atoms, wherein R² is optionally alkyl, or

b) form another bond optionally between carbon atoms binding with the members of said pairs;

R⁷ is hydrogen atom, hydroxy, protected hydroxy or alkyloxy, or may form oxo with R¹;

R⁸ and R⁹ each independently show hydrogen atom or hydroxy;

R¹⁰ is hydrogen atom, alkyl, alkyl substituted by one or more hydroxy, alkenyl, alkenyl substituted by one or more hydroxy or alkyl substituted by oxo;

X is oxo, (hydrogen atom, hydroxy), (hydrogen atom, hydrogen atom), or a group of the formula —CH₂O—;

Y is oxo, (hydrogen atom, hydroxy), (hydrogen atom, hydrogen atom), or a group of the formula N—NR¹¹R¹² or N—OR¹³;

R¹¹ and R¹² each independently show hydrogen atom, alkyl, aryl or tosyl;

R¹³, R¹⁴, R¹⁵, R¹⁶, R¹⁷, R¹⁸, R¹⁹, R²² and R²³ each independently show hydrogen atom or alkyl;

R²⁴ is an optionally substituted ring that may contain one or more hetero atom(s); and n is 1 or 2,

in addition to the meaning noted above, Y, R¹⁰ and R²³ may have, together with the carbon atom they bind with, a saturated or unsaturated 5 or 6-membered heterocyclic group containing nitrogen atom, sulfur atom and/or oxygen atom, the heterocyclic group being optionally substituted by one or more group(s) selected from alkyl, hydroxy, alkyloxy, benzyl, a group of the formula —CH₂Se(C₆H₅), and alkyl substituted by one or more hydroxy, or its pharmaceutically acceptable salt.

Further, the present invention relates to a method for treating ocular inflammatory diseases, comprising topical administration of an agent for topical ophthalmic treatment comprising a tricyclo compound as shown by the above general formula (I) or its pharmaceutically acceptable salt to the eye of a human in need of the treatment of ocular inflammatory diseases in the concentration of 0.01%-0.1%.

Further, the present invention relates to a use of a tricyclo compound as shown by the above general formula (I) or its pharmaceutically acceptable salt for manufacturing an agent for topical ophthalmic treatment of a human for treating ocular inflammatory diseases, wherein said agent comprises a tricyclo compound in the concentration of 0.01%-0.1%.

In the general formula (I), R²⁴ is preferably, for example, cyclo(C₅-C₇)alkyl optionally having one or more suitable substituents, such as the following.

(a) 3,4-dioxocyclohexyl,

(b) 3-R²⁰-4-R²¹-cyclohexyl, wherein R²⁰ is hydroxy, alkyloxy or —OCH₂OCH₂CH₂OCH₃, and R²¹ is hydroxy, —OCN, alkyloxy, heteroaryloxy having suitable substituent, —OCH₂OCH₂CH₂OCH₃, protected hydroxy, chloro, bromo, iodo, aminooxalyloxy, azide, p-tolyloxythiocarbonyloxy, or R²⁵R²⁶CHCOO— (wherein R²⁵ is hydroxy optionally protected where desired or protected amino, and R²⁶ is hydrogen atom or methyl, or R²⁰ and R²¹ in combination form an oxygen atom of epoxide ring); or

(c) cyclopentyl wherein cyclopentyl is substituted by methoxymethyl, optionally protected hydroxymethyl where desired, acyloxymethyl (wherein acyl moiety is optionally quaternized dimethylamino or optionally esterified carboxy), one or more optionally protected amino and/or hydroxy, or aminooxalyloxymethyl. Preferable examples include 2-formyl-cyclopentyl.

The definition of each symbol used in the formula (I), specific examples thereof and preferable embodiments thereof will be explained in detail in the following.

“Lower” means a group having 1 to 6 carbon atoms unless otherwise indicated.

Preferable examples of the alkyl moiety of“alkyl” and “alkyloxy” include linear or branched hydrocarbon residue, such as lower alkyl (e.g., methyl, ethyl, propyl, isopropyl, butyl, isobutyl, pentyl, neopentyl, hexyl and the like).

Preferable examples of “alkenyl” include linear or branched hydrocarbon residue having one double bond, such as lower alkenyl (e.g., vinyl, propenyl (e.g., allyl and the like), butenyl, methylpropenyl, pentenyl, hexenyl and the like).

Preferable examples of “aryl” include phenyl, tolyl, xylyl, cumenyl, mesityl, naphthyl and the like.

Preferable examples of the protective group for “protected hydroxy” and “protected amino” include 1-(loweralkylthio) (lower) alkyl such as lower alkylthiomethyl (e.g., methylthiomethyl, ethylthiomethyl, propylthiomethyl, isopropylthiomethyl, butylthiomethyl, isobutylthiomethyl, hexylthiomethyl and the like), with more preference given to C₁-C₄ alkylthiomethyl and most preference given to methylthiomethyl;

tri-substituted silyl such as tri (lower) alkylsilyl (e.g., trimethylsilyl, triethylsilyl, tributylsilyl, tert-butyl dimethylsilyl, tri-tert-butylsilyl and the like), and lower alkyldiarylsilyl (e.g., methyldiphenylsilyl, ethyldiphenylsilyl, propyldiphenylsilyl, tert-butyldiphenylsilyl and the like), with more preference given to tri(C₁-C₄) alkylsilyl and C₁-C₄ alkyldiphenylsilyl, and most preference given to tert-butyl-dimethylsilyl and tert-butyldiphenylsilyl;

acyl such as aliphatic acyl, aromatic acyl and aliphatic acyl substituted by aromatic group, which are derived from carboxylic acid, sulfonic acid and carbamic acid; and the like.

The aliphatic acyl is exemplified by lower alkanoyl optionally having one or more suitable substituent(s) (e.g., carboxy) such as formyl, acetyl, propionyl, butyryl, isobutyryl, valeryl, isovaleryl, pivaloyl, hexanoyl, carboxyacetyl, carboxypropionyl, carboxybutyryl, carboxyhexanoyl and the like;

cyclo(lower)alkyloxy(lower)alkanoyl optionally having one or more suitable substituent(s) (e.g., lower alkyl) such as cyclopropyloxyacetyl, cyclobutyloxypropionyl, cycloheptyloxybutyryl, mentyloxyacetyl, mentyloxypropionyl, mentyloxybutyryl, mentyloxypentanoyl, mentyloxyhexanoyl and the like;

camphorsulfonyl;

lower alkylcarbamoyl having one or more suitable substituent(s) such as carboxy or protected carboxy and the like, such as carboxy(lower)alkylcarbamoyl (e.g., carboxymethylcarbamoyl, carboxyethylcarbamoyl, carboxypropylcarbamoyl, carboxybutylcarbamoyl, carboxypentylcarbamoyl, carboxyhexylcarbamoyl) and

tri(lower)alkylsilyl(lower)alkyloxycarbonyl(lower)alkylcarbamoyl (e.g., trimethylsilylmethoxycarbonylethylcarbamoyl, trimethylsilylethoxycarbonylpropylcarbamoyl, triethylsilylethoxycarbonylpropylcarbamoyl, tert-butyl dimethylsilylethoxycarbonylpropylcarbamoyl, trimethylsilylpropoxycarbonylbutylcarbamoyl).

Aromatic acyl is exemplified by aroyl optionally having one or more suitable substituent(s) (e.g., nitro), such as benzoyl, toluoyl, xyloyl, naphthoyl, nitrobenzoyl, dinitrobenzoyl, nitronaphthoyl and the like; and

arenesulfonyl optionally having one or more suitable substituent(s) (e.g., halogen), such as benzenesulfonyl, toluenesulfonyl, xylenesulfonyl, naphthalenesulfonyl, fluorobenzenesulfonyl, chlorobenzenesulfonyl, bromobenzenesulfonyl, iodobenzenesulfonyl and the like.

The aliphatic acyl substituted by aromatic group may be, for example, ar(lower)alkanoyl optionally having one or more suitable substituent(s) (e.g., lower alkyloxy or trihalo(lower)alkyl and the like), wherein specific examples are phenylacetyl, phenylpropionyl, phenylbutyryl, 2-trifluoromethyl-2-methoxy-2-phenylacetyl, 2-ethyl-2-trifluoromethyl-2-phenylacetyl, 2-trifluoromethyl-2-propoxy-2-henylacetyl and the like.

Of the above-mentioned acyl, more preferable acyl includes C₁-C₄ alkanoyl optionally having carboxy, cyclo(C₅-C₆)alkyloxy(C₁-C₄)alkanoyl having two (C₁-C₄)alkyl in the cycloalkyl moiety, camphorsulfonyl, carboxy (C₁-C₄)alkylcarbamoyl, tri(C₁-C₄)alkylsilyl(C₁-C₄)alkyloxycarbonyl(C₁-C₄)alkylcarbamoyl, benzoyl, optionally having one or two nitro groups, and benzenesulfonyl having halogen, phenyl(C₁-C₄)alkanoyl having C₁-C₄ alkyloxy and trihalo(C₁-C₄)alkyl. Of these, most preferred are acetyl, carboxypropionyl, mentyloxyacetyl, camphorsulfonyl, benzoyl, nitrobenzoyl, dinitrobenzoyl, iodobenzenesulfonyl, 2-trifluoromethyl-2-methoxy-2-phenylacetyl and the like.

Preferable examples of the “heterocyclic group consisting of saturated or unsaturated 5 or 6-membered ring having nitrogen atom, sulfur atom and/or oxygen atom” are pyrolyl, tetrahydrofuryl and the like.

The “heteroaryl optionally having a suitable substituent moiety” of the “heteroaryloxy optionally having a suitable substituent” is that exemplified for R¹ of the compound of the formula I of EP-A-532,088, with preference given to 1-hydroxyethylindol-5-yl (incorporated herein by reference in its entirety).

The tricyclo compound (I) used in the present invention is described in the publications EP-A-184162, EP-A-323042, EP-A-423714, EP-A-427680, EP-A-465426, EP-A-480623, EP-A-532088, EP-A-532089, EP-A-569337, EP-A-626385, WO89/05303, WO93/05058, WO96/31514, WO91/13889, WO91/19495, WO93/5059 and the like. The disclosures of each of these publications are incorporated herein by reference in their entirety.

In particular, the compounds called FR900506 (=FK506), FR900520 (Ascomycin), FR900523 and FR900525 produced by the genus Streptomyces, such as Streptomyces tsukubaensis, No. 9993 (depository: National Institute of Advanced Industrial Science and Technology, International Patent Organism Depositary, Central 6, 1-1, Higashi 1-chome, Tsukuba-shi, Ibaraki-ken, Japan (formerly: Fermentation Research Institute, Agency of Industrial Science and Technology, the Ministry of International Trade and Industry), date of deposit_FOct. 5, 1984, deposit number: FERM BP-927) or Streptomyces hygroscopicus subsp. Yakushimaensis, No. 7238 (depository: National Institute of Advanced Industrial Science and Technology, International Patent Organism Depositary, Central 6, 1-1, Higashi 1-chome, Tsukuba-shi, Ibaraki-ken, Japan (formerly: Fermentation Research Institute, Agency of Industrial Science and Technology, the Ministry of International Trade and Industry), date of deposit: Jan. 12, 1985, deposit number: FERM BP-928 (EP-A-0184162)), and the compound of the following formula, FK506 (general name Tacrolimus) is a representative compound.

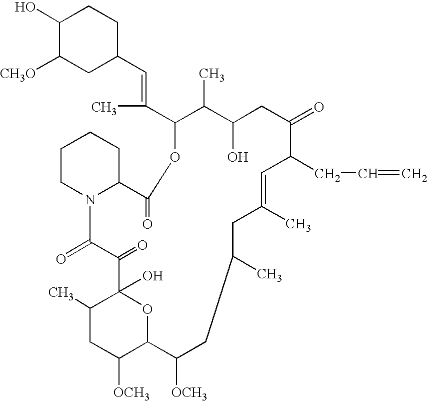

Chemical name: 17-allyl-1,14-dihydroxy-12-[2-(4-hydroxy-3-methoxycyclohexyl)-1 -methylvinyl]-23,25-dimethoxy-13,19,21,27-tetramethyl-11,28-dioxa-4-azatricyclo [22.3.1.0^{4,9}]octacos-18-ene-2,3,10,16-tetraone

Of the tricyclo compounds (I), more preferred is a compound wherein adjacent pairs of R³ and R⁴, and R⁵ and R⁶ each independently form another bond optionally between carbon atoms binding with the members of said pairs;

R⁸ and R ²³ each independently show hydrogen atom;

R⁹ is hydroxy;

R¹⁰ is methyl, ethyl, propyl or allyl;

X is (hydrogen atom, hydrogen atom) or oxo;

Y is oxo;

R¹⁴, R¹⁵, R¹⁶, R¹⁷, R¹⁸, R¹⁹ and R²² each independently show methyl;

R²⁴ is 3-R²⁰-4-R²¹-cyclohexyl,

wherein R²⁰ is hydroxy, alkyloxy or —OCH₂OCH₂CH₂OCH₃, and

R²¹ is hydroxy, —OCN, alkyloxy, heteroaryloxy having suitable substituent, —OCH₂OCH₂CH₂OCH₃, protected hydroxy, chloro, bromo, iodo, aminooxalyloxy, azide, p-tolyloxythiocarbonyloxy or R²⁵R²⁶CHCOO— (wherein R²⁵ is optionally protected hydroxy as desired, or protected amino, and R²⁶ is hydrogen atom or methyl), or R²⁰ and R²¹ in combination form an oxygen atom of epoxide ring; and

n is 1 or 2.

Particularly preferable tricyclo compounds (I) include, besides FK506, Ascomycin derivatives such as halogenated derivative of 33-epi-chloro-33-desoxy Ascomycin described in Example 66a of EP-A-427,680 and the like.

The tricyclo compound (I) and its pharmaceutically acceptable salts are nontoxic. Pharmaceutically acceptable conventional salts are exemplified by salts with inorganic or organic base such as alkali metal salt (e.g., sodium salt, potassium salt and the like), alkaline earth metal salt (e.g., calcium salt, magnesium salt and the like), ammonium salt, and amine salt (e.g., triethylamine salt, N-benzyl-N-methylamine salt and the like).

In the tricyclo compound of the present invention, conformers or one or more pairs of stereoisomers such as optical isomers and geometric isomers due to asymmetric carbon atom and double bond may be present. Such conformers or isomers are also encompassed in the present invention. In addition, the tricyclo compound can form solvates, which are also encompassed in the present invention. Examples of preferable solvates include hydrates and ethanolates.

In the present invention, the ocular inflammatory diseases include ocular inflammatory diseases as expressed in connection with, or as a result of, uveitis, conjunctivitis, cyclitis, scleritis, episcleritis, optic neuritis, retrobulbar optic neuritis, keratitis, blepharitis, corneal ulcer, conjunctival ulcer, etc.; ocular inflammatory diseases caused by ocular disorders such as dry eye, ocular infection, optic nerve disorder, etc.; ocular inflammatory diseases caused by an ophthalmic operation; and ocular inflammatory diseases caused by a physical injury to the eye. Also included in the inflammatory diseases in the present invention are ocular inflammatory diseases of unknown cause, such as chronic nummular keratitis, Thygeson keratitis, progressive Mooren's ulcer, etc.

The present invention also includes the treatment of symptoms caused by the ocular inflammatory diseases including itching, flare, edema, ulcer, etc.

The present agent for topical ophthalmic treatment shows excellent ocular anti-inflammatory effects by topical administration in low doses to the eye of a human suffering from ocular inflammatory diseases. Particularly, the present agent for topical ophthalmic treatment contains a tricyclo compound, as shown by the general formula (I), as the active ingredient in the concentration of 0.01%-0.1%.

Further, the present agent is effective even for subjects in whom conventional anti-inflammatory agents (e.g., steroid, cyclosporins, etc.) show no improving effect.

Furthermore, unlike steroid treatment, the present agent shows ocular anti-inflammatory effects without increasing intraocular pressure, thus reducing the side effects caused by other anti-inflammatory agents. Accordingly, the agent is effective even for subjects for whom other anti-inflammatory agents cannot be used (e.g., steroid contraindication).

The term “treatment” used herein includes any means of control such as prevention, care, relief of the condition, attenuation of the condition, arrest of progression, etc.

The compound of general formula (I) used as the active ingredient of the present invention is administered topically to the eye in the forms of eye drops, eye ointment, etc.

In the case of administering a formulation, the formulation manufactured according to ordinary means can be administered. The form includes all the formulations for topical administration to the eye used in the ophthalmic field such as eye drops, eye ointment, etc. Eye drops are prepared by dissolving the active ingredient in a sterile aqueous solution such as saline, buffering solution, etc., or by combining powder compositions to be dissolved before use. The eye ointment is prepared by mixing the active ingredient into a base. Such formulations can be prepared according to ordinary means.

Eye drops such as the ones as described in EP-A-040679 1 (incorporated herein by reference) are preferred. If desired, additives ordinarily used in eye drops can be added. Such additives include isotonizing agents (e.g., sodium chloride, etc.), buffer agent (e.g., boric acid, sodium monohydrogen phosphate, sodium dihydrogen phosphate, etc.), preservatives (e.g., benzalkonium chloride, benzethonium chloride, chlorobutanol, etc.), and thickeners (e.g., saccharide such as lactose, mannitol, maltose, etc.; e.g., hyaluronic acid or its salt such as sodium hyaluronate, potassium hyaluronate, etc.; e.g., mucopolysaccharide such as chondroitin sulfate, etc.; e.g., sodium polyacrylate, carboxyvinyl polymer, crosslinked polyacrylate, etc.).

Mixing the active ingredient into a base ordinarily used for eye ointment and formulating according to ordinary methods can sterilely prepare the eye ointment. Examples of the base for the eye ointment include petrolatum, selen 50, Plastibase, macrogol, etc., but not limited thereto. Further, in order to increase the hydrophilicity, a surface-active agent can be added. Regarding the eye ointment, the above-mentioned additives such as the preservatives, etc. can be combined, if necessary.

The present agent for topical ophthalmic treatment can be formulated as a sterile unit dose type containing no preservatives.

The amount of administration and the number of administrations of the active ingredient used in the present invention vary according to the sex, age and weight of the human, symptoms to be treated, effects of treatment to be desired, administration methods, period of treatment, etc. Ordinarily, in the case of using the formulation of eye drops for an adult, a formulation containing 0.01%-0.1% of the active ingredient can be instilled several times a day per eye, preferably one to six times, more preferably one to four times, several drops per time, preferably one to four drops. In the case of using the formulation of an eye ointment, the formulation containing 0.01%-0.1% of the active ingredient can be applied several times a day, preferably one to six times, more preferably one to four times. The present agent for topical ophthalmic treatment is very useful especially since it shows sufficient effects by one to four times of ocular instillation or application.

In the present invention, the formulation can include one active ingredient or a combination of two or more active ingredients. In a combination of plural active ingredients, their respective contents can be suitably increased or decreased in consideration of their effects, safety, etc.

Further, the present formulation can suitably include other pharmacologically active ingredients as far as they do not contradict the object of the present invention.

The further details of the present invention will follow with reference to the examples, which, however, are not intended to limit the present invention.

### EXAMPLE 1

Method 1

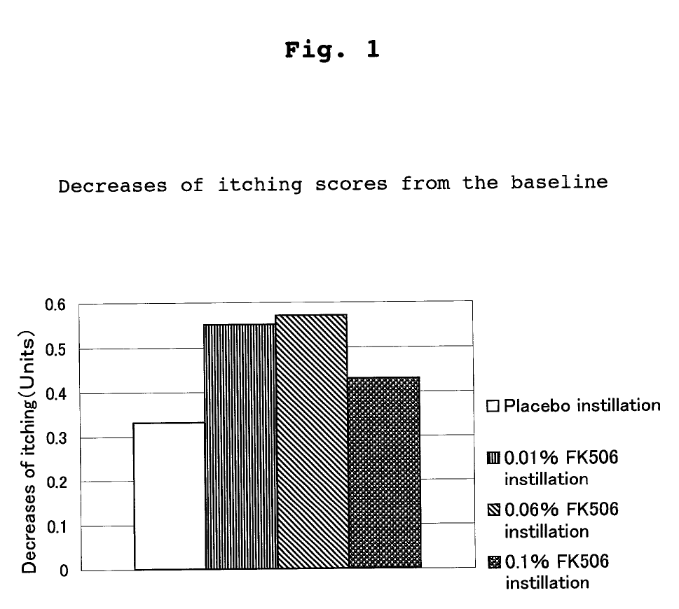

In a total of four groups each having 30 persons, FK506 eye drops (0.01%, 0.06% and 0.1%) were instilled in the respective experimental groups, and placebo was instilled in the control group. Three hours after the ocular instillation, various foreign bodies (cat hair, cat dander, and pollens of a tree, ragweed or grass) were ocularly instilled in both the experimental groups and the control group, thus causing inflammations. Five minutes later, itching on the eye was graded according to five-rank scores (0-4). The decreases from the score (baseline) in instilling only foreign bodies were calculated. These results are shown in FIG. 1.

As shown in FIG. 1, the decreases of itching were greater in the experimental groups instilled with 0.01%, 0.06% and 0.1% of FK506 eye drops than in the control groups instilled with placebo. These results confirmed that the instillation of FK506 eye drops in a low dose of 0.01%-0.1% shows ocular anti-inflammatory effects.

### EXAMPLE 2

FK506 was ocularly instilled in the subjects once a day for one week, and the same amount of placebo was ocularly instilled in the control group. At 16 hours after the final ocular instillation, various foreign bodies (cat hair, cat dander, and pollens of a tree, ragweed or grass) were ocularly instilled in both the experimental groups and the control group, thus causing the inflammations. Ten minutes later, conjunctival hyperemia and chemosis were graded according to five-rank scores (0-4). The changes from the score (baseline) in instilling only foreign bodies were calculated. These results are shown in Tables 1 and 2.

TABLE 1
conjunctival hyperemia

| | | Changes of conjunctival hyperemia scores from |
|---|---|---|
| | Number of | the baseline, |
| Groups | subjects | mean ± S.E. |
| Control | 50 | −0.17 ± 0.07 |
| (placebo) | | |
| 0.1% FK506 | 53 | −0.51 ± 0.07** |
| eye drops | | |

**p < 0.01

TABLE 2
chemosis

| | Number of | Changes of chemosis scores from the |
|---|---|---|
| Groups | subjects | baseline, mean ± S.E. |
| Control | 50 | 0.12 ± 0.07 |
| (placebo) | | |
| 0.1% FK506 | 53 | −0.30 ± 0.07** |
| eye drops | | |

**p < 0.001

As shown in Tables 1 and 2, compared to the control group instilled with placebo, the instillation of 0.1% FK506 eye drops clearly decreased the scores of both conjunctival hyperemia and chemosis. These results confirmed that the instillation of FK506 eye drops in a low dose shows the ocular anti-inflammatory effects (antiedemic effect and anti-flare effect) for at least 16 hours.

The following are the examples of the instillation of FK506 eye drops in a low dose in patients having various ocular inflammatory diseases.

### EXAMPLE 3

A patient suffering from progressive corneal ulcer caused by pemphigoid was instilled with 0.06% FK506 eye drops three times a day. As a result, significant improving effects were observed and such effects were maintained at 43 weeks later.

### EXAMPLE 4

A patient suffering from progressive Mooren's ulcer was instilled with 0.06% FK506 eye drops three times a day. As a result, significant improving effects were observed and such effects were maintained at 41 weeks later.

### EXAMPLE 5

A patient suffering from chronic nummular keratitis was instilled with 0.06% FK506 eye drops three times a day. As a result, significant improving effects were observed within two weeks and such effects were maintained at 43 weeks later.

### EXAMPLE 6

A patient suffering from Thygeson keratitis, for whom no conventional therapy is available (the topical administration of corticosteroid shows no improving effect, or corticosteroid cannot be used for the topical or systemic administration) and the ocular instillation of cyclosporin A shows no improving effect, was instilled with 0.06% FK506 eye drops three times a day. As a result, significant improving effects were observed within three weeks and such effects were maintained at 41 weeks later.

### EXAMPLE 7

A patient having undergone a penetrating keratoplasty and having a history of steroid glaucoma and also having a history of chronic rejection despite the ocular instillation of cyclosporin A was instilled with 0.06% FK506 eye drops three times a day. As a result, the progression of inflammations caused by injury was arrested and such effects were maintained at 34 weeks later. Besides, no intraocular pressure increase was observed.

### EXAMPLE 8

A patient suffering from blepharokeratoconjunctivitis, for whom no conventional therapy is available (the topical administration of corticosteroid shows no improving effect, or corticosteroid cannot be used for the topical or systemic administration) and the ocular instillation of cyclosporin A shows no improving effect, was instilled with 0.06% FK506 eye drops three times a day. As a result, significant improving effects were observed within two weeks and such effects were maintained at 18 weeks later.

### EXAMPLE 9

A patient performed a penetrating keratoplasty due to keratoconus and having a history of refractoriness to the topical cyclosporins A, for whom no conventional therapy is available (the topical administration of corticosteroid shows no improving effect, or corticosteroid cannot be used for the topical or systemic administration), was instilled with 0.06% FK506 eye drops three times a day. As a result, significant improving effects were observed and such effects were maintained at 25 weeks later.

As shown in the foregoing examples 3-9, it was confirmed that the topical instillation of FK506 eye drops in a low dose in the eye of a human having various ocular inflammatory diseases shows anti-inflammatory effects.

It was further confirmed that the present agent for topical ophthalmic treatment is effective even for a subject in whom conventional anti-inflammatory agents show no improving effect (e.g., steroid, cyclosporins, etc.), and that the present agent shows anti-inflammatory effects in a subject for whom other anti-inflammatory agents cannot be used (e.g., steroid contraindication).

This application is based on application Ser. No. 60/283,169 filed in United States of America, the entire contents of which are incorporated herein by reference.

Obviously, numerous modifications and variations of the present invention are possible in light of the above teachings. It is therefore to be understood that within the scope of the appended claims, the invention may be practiced otherwise than as specifically described herein.

1. A method for treating one or more ocular inflammatory diseases, comprising
topically administering an agent for topical ophthalmic treatment comprising a tricyclo compound of formula (I), or a pharmaceutically acceptable salt thereof, in a concentration of from 0.01% to 0.1% to the eye of a human in need of treatment of ocular inflammatory disease: 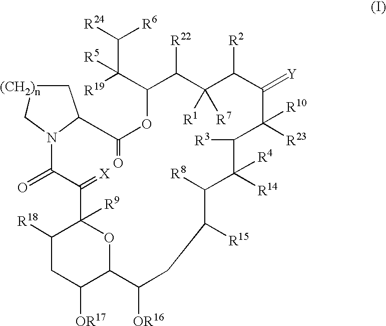
wherein adjacent pairs of R¹ and R², R³ and R⁴, and R⁵ and R⁶ each independently may be
a) two adjacent hydrogen atoms, wherein R² is optionally alkyl, or
b) may form a bond between carbon atoms bound with the members of said pairs;
R⁷ is a hydrogen atom, a hydroxy group, a protected hydroxy group, an alkyloxy group, or may form an oxo group with R¹;
R⁸ and R⁹ may each independently be a hydrogen atom or a hydroxy group;
R¹⁰ is a hydrogen atom, an alkyl group, an alkyl group substituted by one or more hydroxy groups, an alkenyl group, an alkenyl group substituted by one or more hydroxy groups or an alkyl group substituted by an oxo group;
X is an oxo group, a hydrogen atom and a hydroxy group, a hydrogen atom and a hydrogen atom, or X combined with an adjacent O can be a group of formula —CH₂O—;
Y is an oxo group, a hydrogen atom and a hydroxy group, a hydrogen atom and a hydrogen atom, a group of formula N—NR¹¹R¹², or a group of formula N—OR¹³;
R¹¹ and R¹² can each independently be a hydrogen atom, an alkyl group, an aryl group or a tosyl group;
R¹³, R¹⁴, R¹⁵, R¹⁶, R¹⁷, R¹⁸, R¹⁹, R²² and R²³ can each independently be a hydrogen atom or an alkyl group;
R²⁴ is an optionally substituted ring that may contain one or more hetero atoms;
n is 1 or 2; and
Y, R¹⁰ and R²³ may, together with a carbon atom, be a saturated or unsaturated 5 or 6-membered heterocyclic group containing one or more nitrogen atoms, sulfur atoms, oxygen atoms or combinations thereof, the heterocyclic group optionally substituted by one or more groups selected from the group consisting of an alkyl group, a hydroxy group, an alkyloxy group, a benzyl group, a group of formula —CH₂Se(C₆H₅), and an alkyl group substituted by one or more hydroxy groups.

2. The method of claim 1, wherein the tricyclo compound is FK506.

3. The method of claim 1, wherein the agent is topically administered one to four times a day.

4. The method of claim 1, wherein the agent for topical ophthalmic treatment is an eye drop or eye ointment.

5. The method of claim 1, wherein the ocular inflammatory diseases are selected from the group consisting of uveitis, conjunctivitis, cyclitis, scleritis, episcleritis, optic neuritis, retrobulbar optic neuritis, keratitis, blepharitis, corneal ulcer, conjunctival ulcer, an ocular inflammatory disease caused by an ocular disorder, an ocular inflammatory disease after an ophthalmic operation, an ocular inflammatory disease caused by a physical injury and symptoms thereof.

6. The method of claim 1, wherein itching on the eye is treated.

7. The method of claim 1, wherein a flare on the eye is treated.

8. The method of claim 1, wherein an edema on the eye is treated.

9. The method of claim 1, wherein an ulcer on the eye is treated.

10. The method of claim 1, comprising administering the agent to a human in whom one or more other ocular anti-inflammatory agents are not effective.

11. The method of claim 10, wherein the other ocular anti-inflammatory agents are cyclosporins, steroid drugs or mixtures thereof.

12. The method of claim 1, comprising administering the agent to a human in whom one or more other ocular anti-inflammatory agents can not be administered.

13. The method of claim 12, wherein the other ocular anti-inflammatory agents are steroid drugs.

14. An agent for topical ophthalmic treatment of one or more ocular inflammatory diseases of a human, comprising a tricyclo compound of formula (I) or a pharmaceutically acceptable salt thereof, as an active ingredient in a concentration of from 0.01% to 0.1%: 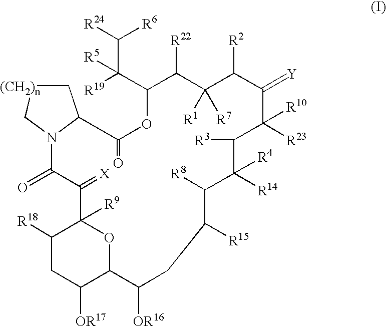
wherein adjacent pairs of R¹ and R², R³ and R⁴, and R⁵ and R⁶ each independently may be
a) two adjacent hydrogen atoms, wherein R² is optionally alkyl, or
b) may form a bond between carbon atoms bound with the members of said pairs;
R⁷ is a hydrogen atom, a hydroxy group, a protected hydroxy group, an alkyloxy group, or may form an oxo group with R¹;
R⁸ and R⁹ may each independently be a hydrogen atom or a hydroxy group;
R¹⁰ is a hydrogen atom, an alkyl group, an alkyl group substituted by one or more hydroxy groups, an alkenyl group, an alkenyl group substituted by one or more hydroxy groups or an alkyl group substituted by an oxo group;
X is an oxo group, a hydrogen atom and a hydroxy group, a hydrogen atom and a hydrogen atom, or X combined with an adjacent O can be a group of formula —CH₂O—;
Y is an oxo group, a hydrogen atom and a hydroxy group, a hydrogen atom and a hydrogen atom, a group of formula N—NR¹¹R¹², or a group of formula N—OR¹³;
R¹¹ and R¹² can each independently be a hydrogen atom, an alkyl group, an aryl group or a tosyl group;
R¹³, R¹⁴, R¹⁵, R¹⁶, R¹⁷, R¹⁸, R¹⁹, R²² and R²³ can each independently be a hydrogen atom or an alkyl group;
R²⁴ is an optionally substituted ring that may contain one or more hetero atoms;
n is 1 or 2; and
Y, R¹⁰ and R²³ may, together with a carbon atom, be a saturated or unsaturated 5 or 6-membered heterocyclic group containing one or more nitrogen atoms, sulfur atoms, oxygen atoms or combinations thereof, the heterocyclic group optionally substituted by one or more groups selected from the group consisting of an alkyl group, a hydroxy group, an alkyloxy group, a benzyl group, a group of formula —CH₂Se(C₆H₅), and an alkyl group substituted by one or more hydroxy groups.

15. A method of preparing an agent for topical ophthalmic treatment of one or more ocular inflammatory diseases of a human, said method comprising
mixing a tricyclo compound of formula (I) or a pharmaceutically acceptable salt thereof, with a pharmaceutically acceptable carrier or excipient, wherein said agent comprises said tricyclo compound in a concentration of from 0.01% to 0.1%: 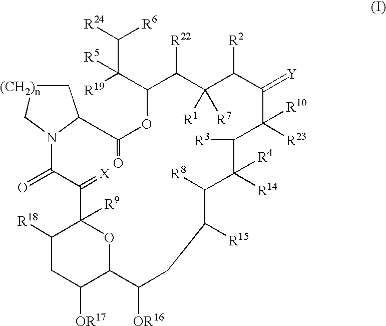
wherein adjacent pairs of R¹ and R², R³ and R⁴, and R⁵ and R⁶ each independently may be
a) two adjacent hydrogen atoms, wherein R² is optionally alkyl, or
b) may form a bond between carbon atoms bound with the members of said pairs;
R⁷ is a hydrogen atom, a hydroxy group, a protected hydroxy group, an alkyloxy group, or may form an oxo group with R¹;
R⁸ and R⁹ may each independently be a hydrogen atom or a hydroxy group;
R¹⁰ is a hydrogen atom, an alkyl group, an alkyl group substituted by one or more hydroxy groups, an alkenyl group, an alkenyl group substituted by one or more hydroxy groups or an alkyl group substituted by an oxo group;
X is an oxo group, a hydrogen atom and a hydroxy group, a hydrogen atom and a hydrogen atom, or X combined with an adjacent O can be a group of formula —CH₂O—;
Y is an oxo group, a hydrogen atom and a hydroxy group, a hydrogen atom and a hydrogen atom, a group of formula N—NR¹¹R¹², or a group of formula N—OR¹³;
R¹¹ and R¹² can each independently be a hydrogen atom, an alkyl group, an aryl group or a tosyl group;
R¹³, R¹⁴, R¹⁵, R¹⁶, R¹⁷, R¹⁸, R¹⁹, R²² and R²³ can each independently be atom or an alkyl group;
R²⁴ is an optionally substituted ring that may contain one or more hetero atoms;
n is 1 or 2; and
Y, R¹⁰ and R²³ may, together with a carbon atom, be a saturated or unsaturated 5 or 6-membered heterocyclic group containing one or more nitrogen atoms, sulfur atoms, oxygen atoms or combinations thereof, the heterocyclic group optionally substituted by one or more groups selected from the group consisting of an alkyl group, a hydroxy group, an alkyloxy group, a benzyl group, a group of the formula —CH₂Se(C₆H₅), and an alkyl group substituted by one or more hydroxy groups.
